(19) United States
(12) Patent Application Publication
(10) Pub. No.: US 2018/0193234 A1
(43) Pub. Date: Jul. 12, 2018

(54) OIL-IN-WATER EMULSION AND METHOD

(71) Applicants: Dow Corning (China) Holding Co., Ltd., Shanghai (CN); Dow Corning Corporation, Midland, MI (US)

(72) Inventors: Severine CAUVIN, MONS (BE); Li DING, Shanghai (CN); Sophie HANSSENS, Chastre (BE); Xinyu SHI, Shanghai (CN); Stephane UGAZIO, Soignies (BE)

(21) Appl. No.: 15/741,014

(22) PCT Filed: Jul. 2, 2015
(86) PCT No.: PCT/CN2015/083175

§ 371 Date: Dec. 29, 2017

(51) Int. Cl. A61K 8/06; A61K 8/31; A61K 8/892; A61K 8/92; A61K 8/46; A61Q 19/00

(52) U.S. Cl. CPC A61K 8/062; A61K 8/31; A61K 8/892; A61K 2800/412; A61K 8/466; A61Q 19/00; A61K 8/922

(57) ABSTRACT

A method of preparing an oil-in-water emulsion comprises combining (A) an organopolysiloxane including at least two silicon-bonded hydrolysable or hydroxyl groups and (B) an organic oil to give a mixture. The method further comprises combining the mixture, (C) an aqueous medium and (D) a surfactant to form an initial emulsion. Finally, the method comprises contacting the organopolysiloxane (A) with (D¹) an organic acid catalyst to polymerize the organopolysiloxane (A) to give (A¹) a high molecular weight organopolysiloxane and the oil-in-water emulsion. A personal care composition comprising the oil-in-water emulsion and a personal care ingredient is also disclosed.

### TECHNICAL FIELD

The present invention generally relates to a method of preparing an oil-in-water emulsion and, more specifically, to a method of preparing an oil-in-water emulsion comprising a high molecular weight organopolysiloxane, its method of preparation, and personal care compositions including such oil-in-water emulsions.

### BACKGROUND

Organopolysiloxanes are well known in the art and have numerous end use applications, including use in compositions for personal care. For example, organopolysiloxanes are utilized in various compositions for personal care and contacting skin, hair, nails, mucosa, etc. Commonly, these compositions are waterborne, and organopolysiloxanes do not readily disperse or solubilize in water. As such, organopolysiloxanes are commonly delivered via emulsions.

Silicone emulsions can be made by various processes, such as mechanical emulsification or emulsion polymerization. Mechanical emulsification entails the homogenization of an oil phase, e.g. a silicone polymer, and an aqueous phase to form a homogeneous emulsion. Mechanical emulsification typically requires considerable amount of energy input to reduce average particle sizes, and there are limitations regardless of this energy input.

Emulsion polymerization, on the other hand, involves the emulsification and polymerization of reactive monomers, oligomers, and/or polymers in water. Emulsion polymerization is commonly less energy demanding than mechanical emulsification. However, even with emulsion polymerization, it is difficult to control average particle sizes.

### BRIEF SUMMARY OF THE INVENTION

The present invention provides a method of preparing an oil-in-water emulsion. The method comprises combining (A) an organopolysiloxane including at least two silicon-bonded hydrolysable or hydroxyl groups and (B) an organic oil to give a mixture. The method further comprises combining the mixture, (C) an aqueous medium and (D) a surfactant to form an initial emulsion. The mixture is a discontinuous phase in the aqueous medium (C) of the initial emulsion. Finally, the method comprises contacting the organopolysiloxane (A) with (D¹) an organic acid catalyst, which is the same as or different from the surfactant (D), to polymerize the organopolysiloxane (A) in the discontinuous phase of the initial emulsion to give (A¹) a high molecular weight organopolysiloxane, thereby preparing the oil-in-water emulsion.

The oil-in-water emulsion formed in accordance with the method is provided. Further, a composition for personal care comprising the oil-in-water emulsion and at least one personal care ingredient is provided. Finally, the present invention provides a method of preparing the composition for personal care. The method of preparing the composition comprises preparing the oil-in-water emulsion as described above. This method further comprises combining the oil-in-water emulsion with at least one personal care ingredient to give the composition.

### DETAILED DESCRIPTION

The present invention provides a method of preparing an oil-in-water emulsion and the oil-in-water emulsion prepared thereby. The oil-in-water emulsion has excellent physical properties and is suitable for use in diverse applications. As described in greater detail below, the oil-in-water emulsion is particularly well suited for use in compositions for personal care. A composition for personal care including the oil-in-water emulsion, a method for preparing the composition, and the composition prepared thereby are also described below.

The term “substantial” or “substantially” as used herein to describe any substantially linear organopolysiloxane means that in relation to the notation of MDTQ of an organopolysiloxane, there is less than 5 mole % or less than 2 mole % of the units T and/or Q. The M, D, T, Q designate one (Mono), two (Di), three (Tri), or four (Quad) oxygen atoms covalently bonded to a silicon atom that is linked into the rest of the molecular structure. The M, D, T and Q units are typically represented as RᵤSiO(_{4-u)/2}, where u is 3, 2, 1, and 0 for M, D, T, and Q, respectively, and R is a substituted or unsubstituted hydrocarbon group.

The term “about” as used herein serves to reasonably encompass or describe minor variations in numerical values measured by instrumental analysis or as a result of sample handling. Such minor variations may be in the order of plus or minus 0% to 10% or plus or minus 0% to 5% of the numerical values.

The term “branched” as used herein describes a polymer with more than two end groups.

The term “comprising” is used herein in its broadest sense to mean and to encompass the notions of “include” and “consist of.”

The term “ambient temperature” or “room temperature” refers to a temperature between about 20° C. and about 30° C. Usually, room temperature ranges from about 20° C. to about 25° C.

The use of “for example” or “such as” to list illustrative examples does not limit to only the listed examples. Thus, “for example” or “such as” means “for example, but not limited to” or “such as, but not limited to” and encompasses other similar or equivalent examples.

The term “substituted” as used in relation to another group, for example, a hydrocarbon group, means, unless indicated otherwise, one or more hydrogen atoms in the hydrocarbon group has been replaced with another substituent. Examples of such substituents include, for example, halogen atoms such as chlorine, fluorine, bromine, and iodine; halogen atom containing groups such as chloromethyl, perfluorobutyl, trifluoroethyl, and nonafluorohexyl; oxygen atoms; oxygen atom containing groups such as (meth)acrylic and carboxyl; nitrogen atoms; nitrogen atom containing groups such as amines, amino-functional groups, amido-functional groups, and cyano-functional groups; sulphur atoms; and sulphur atom containing groups such as mercapto groups.

All viscosity measurements referred to herein were measured at 25° C. unless otherwise indicated.

An organopolysiloxane is intended to mean a polymer comprising multiple organosiloxane or polyorganosiloxane groups per molecule. Organopolysiloxane is intended to include polymers substantially containing only organosiloxane or polyorganosiloxane groups in the polymer chain, and polymers where the backbone contains both organosiloxane and/or polyorganosiloxane groups and organic polymer groups in the polymer chain. Such polymers may be homopolymers or copolymers, including, for example, block copolymers and random copolymers.

While the organopolysiloxane polymer has a substantially organopolysiloxane molecular chain, the organopolysiloxane polymer may alternatively contain a block copolymeric backbone comprising at least one block of siloxane groups and an organic component comprising any suitable organic based polymer backbone, for example, the organic polymer backbone may comprise, for example, polystyrene and/or substituted polystyrenes such as poly(α-methylstyrene), poly(vinylmethylstyrene), dienes, poly(p-trimethylsilylstyrene) and poly(p-trimethylsilyl-α-methylstyrene). Other organic components which may be incorporated in the polymeric backbone may include acetylene terminated oligophenylenes, vinylbenzyl terminated aromatic polysulphones oligomers, aromatic polyesters, aromatic polyester based monomers, polyalkylenes, polyurethanes, aliphatic polyesters, aliphatic polyamides and aromatic polyamides and the like.

The method comprises combining (A) an organopolysiloxane including at least two silicon-bonded hydrolysable or hydroxyl groups and (B) an organic oil to give a mixture.

The organopolysiloxane (A) may be linear, branched, or resinous. Combinations of different organopolysiloxanes having one or more different structures may be utilized in concert as the organopolysiloxane (A). In various embodiments, the organopolysiloxane (A) has the formula:

(R_{3-w}X_{w}SiO_{1/2})ₐ(R₂₋ₓXₓSiO_{2/2})_{b}(R_{1-y}X_{y}SiO_{3/2})_{c}(SiO_{4/2})_{d},

wherein each R is independently a substituted or unsubstituted hydrocarbyl group, each X is independently selected from R and a hydrolysable or hydroxyl group, w is an integer from 0 to 3, x is an integer from 0 to 2, y and 0 or 1, with the proviso that (w+x+y)≥2 and (a+b+c+d)=1. In certain embodiments, b>(a+c+d). In these or other embodiments, 0≤(c+d)≤0.10. Because (w+x+y)≥2, the organopolysiloxane (A) includes at least two silicon-bonded hydrolysable or hydroxyl groups. Such groups may be independently selected, and the organopolysiloxane (A) may include a combination of hydrolysable and hydroxyl groups.

In certain embodiments, the organopolysiloxane (A) is substantially linear and comprises D units capped with M units, although even in such embodiments the organopolysiloxane (A) may include at least some branching attributable to the presence of at least some T and/or Q units. Alternatively, the organopolysiloxane (A) may be linear. In these or other embodiments, the silicon-bonded hydrolysable or hydroxyl groups may be independently terminal and/or pendent in the organopolysiloxane (A). When the organopolysiloxane (A) is substantially linear or linear, the silicon-bonded hydrolysable or hydroxyl groups are typically terminal, e.g. at opposite terminal locations. One terminal may include more than one silicon-bonded hydrolysable or hydroxyl groups, which may be independently selected, even when the silicon-bonded hydrolysable or hydroxyl groups are at opposite terminal locations in the organopolysiloxane (A).

When the organopolysiloxane (A) is substantially linear or linear, the organopolysiloxane (A) may have, for example, a general formula (1):

X¹-A-X² (1),

wherein X¹ and X² are independently selected from silicon-containing groups including at least one silicon-bonded hydrolysable or hydroxyl group and A represents a polymer chain. Specific examples of X¹ and/or X² groups incorporating silicon-bonded hydrolysable or hydroxyl groups include groups terminating with:
—Si(OH)₃; —(R^{a})Si(OH)₂; —(R^{a})₂SiOH; —(R^{a})Si(OR^{b})₂; —Si(OR^{b})₃; —(R^{a}₂)SiOR^{b}; or
—(R^{a}₂)Si—R^{c}—SiR^{d}ₚ(OR^{b})₃₋ₚ,
   wherein each R^{a} independently represents a monovalent hydrocarbyl group having from 1 to 8 carbon atoms, for example, an alkyl group such as methyl; R^{b} is an alkyl; and R^{d} is an alkyl or alkoxy group, wherein the alkyl and alkoxy groups have 1 to 6 carbon atoms; R^{c} is a divalent hydrocarbon group having 1 to 8 carbon atoms which may be interrupted by one or more siloxane spacers having 1 to 6 silicon atoms; and p has the value 0, 1 or 2. The organopolysiloxane (A) may include a small amount, for example, less than 20%, of non-reactive terminal groups, e.g. those of the formula R^{a}₃SiO_{1/2}.

In one embodiment, the polymer chain A of general formula (1) above can comprise a polydiorganosiloxane chain comprising siloxane units of formula (2):

—(R²₂SiO)— (2),

wherein each R² is independently an organic group such as a hydrocarbon group having from 1 to 18 carbon atoms, a substituted hydrocarbon group having from 1 to 18 carbon atoms or a hydrocarbonoxy group having 1 to 18 carbon atoms.

Hydrocarbon groups suitable for R² include, for example, methyl, ethyl, propyl, butyl, vinyl, cyclohexyl, phenyl and tolyl groups. Substituted hydrocarbon groups have one or more hydrogen atoms in a hydrocarbon group replaced with another substituent, for example, a halogen atom such as chlorine, fluorine, bromine or iodine, an oxygen atom containing group such as acrylic, methacrylic, alkoxy or carboxyl, a nitrogen atom containing group such as an amino, amido or cyano group, or a sulphur atom containing group such as a mercapto group. Examples of substituted hydrocarbon groups include a propyl group substituted with chlorine or fluorine such as 3,3,3-trifluoropropyl, chlorophenyl, beta-(perfluorobutyl)ethyl or chlorocyclohexyl group. In some embodiments, at least some or all of the R² groups are methyl.

The polydiorganosiloxanes including units of formula (2) can be polydialkylsiloxanes, for example, polydimethylsiloxanes. The polydiorganosiloxane chain comprising units of formula (2) may be homopolymers or copolymers. Mixtures of different polydiorganosiloxanes or units thereof are also suitable. In the case of polydiorganosiloxane copolymers, the polymer chain may comprise a combination of blocks made from chains of units depicted in formula (2).

The polymer chain A may alternatively have a block copolymeric backbone comprising at least one block of siloxane groups of the type depicted in formula (2) above and at least one block comprising any suitable organic polymer chain. Examples of suitable organic polymer chains can be polyacrylic, polyisobutylene and polyether chains.

In specific embodiments, the organopolysiloxane (A) has the formula:

X_{w}R²_{3-w}SiO(SiR²₂O)ₙSiR²_{3-y}X_{y},

wherein X, R², w, n and y are as defined above. One specific example of such an organopolysiloxane when each X is a hydroxyl group, w is 1, y is 1, and all of the R² groups are methyl groups is dimethylhydroxy-terminated polydimethylsiloxane.

Suitable silicon-bonded hydrolysable groups include those which are capable of undergoing hydrolysis in the presence of water and optionally a catalyst to provide silanol (SiOH) groups, or silicon bonded hydroxyl groups. Specific examples of hydrolysable groups include H, a halide group, an alkoxy (—OR³) group, an alkylamino (—NHR³ or —NR³R⁴) group, a carboxy (—OOC—R³) group, an alkyliminoxy (—O—N═CR³R⁴) group, an alkenyloxy (O—C(═CR³R⁴)R⁵) group, or an N-alkylamido (—NR³COR⁴) group, wherein R³, R⁴ and R⁵ are each independently selected from H and a C₁-C₂₂ hydrocarbyl group. When R³, R⁴ and R⁵ are independently C₁-C₂₂ hydrocarbyl groups, R³, R⁴ and R⁵ may be linear, branched, or cyclic (for C₃-C₂₂ hydrocarbyl groups). In addition, R³, R⁴ and R⁵ may independently include one or more heteroatoms, such as N, O, and/or S, within the hydrocarbyl group, and may be substituted or unsubstituted. Typically, R³, R⁴ and R⁵ are each independently selected C₁-C₄ alkyl groups. When the hydrolysable group is the NR³R⁴ group, R³ and R⁴ optionally can be taken together with the N atom to which they are bonded to form a cyclic amino group. Generally, the organopolysiloxane (A) includes silicon-bonded hydroxyl groups rather than silicon-bonded hydrolysable groups. However, the organopolysiloxane (A) may include silicon-bonded hydrolysable groups which are converted to silicon-bonded hydroxyl groups, e.g. in situ, for polymerization. These silanol groups of the organopolysiloxane (A) condense to form siloxane bonds between adjacent molecules of the organopolysiloxane (A), thus polymerizing the organopolysiloxane (A), as described below. This polymerization may be referred to as condensation polymerization.

In various embodiment, the organopolysiloxane (A) has a dynamic viscosity of from 0.02 to 150, alternatively from 0.05 to 5, alternatively from 0.08 to 2.5, Pa·s at 25° C. Methods of measuring dynamic viscosity are well known. Unless otherwise indicated, dynamic viscosity values recited herein are measured via a Brookfield viscometer in accordance with ASTM D4287.

The organic oil (B) is typically a non-reactive or inert, i.e., the organic oil (B) does not participate in any reaction involving the organopolysiloxane (A). The organic oil (B) is generally chosen to have no groups reactive with the organopolysiloxane (A), and typically serves as the carrier or medium for polymerization of the organopolysiloxane (A).

In one embodiment of the present invention, the organic oil (B) is a liquid. The organic oil (B) in liquid form provides advantages that include, among others, the formation of desirable and flowable products for easy handling.

The organic oil (B) may comprise any suitable organic oil or combination of organic oils in accordance with in the methods and emulsions of the present invention. In certain embodiments, suitable organic oils include those which dissolve the organopolysiloxane (A), which typically forms a clear solution, and those which can be combined with the organopolysiloxane (A) to form a homogeneous dispersion without phase separation prior to, during, and/or after the formation of the oil-in-water emulsion. Any of the fluids described as extenders in WO2006/106362, which is incorporated by reference in its entirety, may be used with or as the organic oil (B). The organic oil may be, for example, any one or combination of the following:
hydrocarbon oils, such as mineral oil fractions comprising linear (e.g., n-paraffinic) mineral oils, branched (iso-paraffinic) mineral oils, and/or cyclic (sometimes referred to as naphthenic) mineral oils, the hydrocarbons in the oil fractions comprising from 5 to 25 carbon atoms per molecule, or a liquid linear or branched paraffin containing 12 to 40 carbon atoms;
polyisobutylenes (PIB);
phosphate esters, such as trioctyl phosphate;
polyalkylbenzenes; linear and/or branched alkylbenzenes, such as heavy alkylates, dodecyl benzene and other alkylarenes;
esters of aliphatic monocarboxylic acids;
linear or branched mono unsaturated hydrocarbons, such as linear or branched alkenes or mixtures thereof containing from 8 to 25 carbon atoms; and
natural oils and derivatives thereof.

In one embodiment, the organic oil (B) may include mineral oil fractions, natural oils, alkylcycloaliphatic compounds, alkybenzenes including polyalkylbenzenes, or combinations thereof.

Alkylbenzene compounds suitable for use as the organic oil (B) include, for example, heavy alkylate alkylbenzenes and alkylcycloaliphatic compounds. Heavy alkylate alkylbenzenes include, for example, alkyl substituted aryl compounds which have aryl groups, such as benzene substituted by alkyl and/or other substituents. Additional examples include the extenders described in U.S. Pat. No. 4,312,801, which is incorporated by reference in its entirety.

Any suitable mixture of mineral oil fractions or mineral oil fractions in combination with any other organic oils may be used as the organic oil (B). Additional examples of organic oils include alkylcyclohexanes and paraffinic hydrocarbons (which may be linear, branched, or cyclic). The cyclic paraffinic hydrocarbons may be monocyclic and/or polycyclic hydrocarbons (naphthenics).

In another embodiment, the organic oil (B) may comprise a natural oil. Natural oils are oils that are not derived from petroleum. More specifically, natural oils are derived from animals and/or vegetative matter (including seeds and nuts). Common natural oils include triglycerides of mixtures of fatty acids, particularly mixtures containing some unsaturated fatty acid. Alternatively, the organic oil (B) may be a derivative of a natural oil such as a transesterified vegetable oil, a boiled natural oil, a blown natural oil, or a stand oil (e.g. a thermally polymerized oil). The natural oil may be derived from a variety of sources and may comprise, for example, wheatgerm, sunflower, grapeseed, castor, shea, avocado, olive, soybean, sweet almond, palm, rapeseed, cotton seed, hazelnut, macadamia, jojoba, blackcurrant, evening primrose, and combinations thereof.

Alternatively to the liquids exemplified above, the organic oil (B) may be a solid, such as a wax. When the organic oil (B) comprises a wax, the wax typically has a melting point of from 30 to 100° C. The wax may be, for example, a hydrocarbon wax, such as a petroleum-derived wax; a wax comprising carboxylic esters, such as beeswax, lanolin, tallow, carnauba, candelilla, tribehenin; or a wax derived from plant seeds, fruits, nuts or kernel, including softer waxes referred to as ‘butter,’ such as mango butter, shea butter or cocoa butter. The wax may alternatively be a polyether wax or a silicone wax.

Notably, when the organic oil (B) comprises a mineral oil, the organic oil (B) and the organopolysiloxane (A) are miscible, i.e., the organic oil (B) and the organopolysiloxane (A) form a homogenous mixture. In contrast, when the organic oil (B) comprises a natural oil, the organic oil (B) and the organopolysiloxane (A) are commonly immiscible, i.e., the organic oil (B) and the organopolysiloxane (A) form a heterogeneous mixture. When the organic oil (B) and the organopolysiloxane (A) are immiscible, generation of cyclic compounds, e.g. cyclic siloxanes, is reduced as compared to when the organic oil (B) and the organopolysiloxane (A) are miscible. It certain embodiments, it is desirable to reduce generation of cyclic compounds.

The mixture formed by combining the organopolysiloxane (A) and the organic oil (B) may be heterogeneous or homogenous. The organic oil (B) may solubilize, alternatively partially solubilize, the organopolysiloxane (A). The organic oil (B) may be referred to as a carrier or a solvent depending on whether the organopolysiloxane (A) solubilizes or dissolves in the organic oil (B). The organopolysiloxane (A) and the organic oil (B) may be combined in any manner, e.g. the organopolysiloxane (A) may be disposed in the organic oil (B), or vice versa, with optional mixing or stirring. Typically, the mixture formed by combining the organopolysiloxane (A) and the organic oil (B) is a homogenous solution.

The relative amounts of the organopolysiloxane (A) and the organic oil (B) may vary. For example, the ratio by weight of the organopolysiloxane (A) to the organic oil may be from 1:10 to 10:1, e.g. 1:1, 3:2, 7:3, 4:1, 1:9, 2:3, 3:7, 1:4 or 9:1. Typically, the organopolysiloxane (A) is utilized in a weight excess as compared to the organic oil (B), i.e., the ratio of the organopolysiloxane (A) to organic oil (B) is >1:1, alternatively >2:1.

The method further comprises combining the mixture, (C) an aqueous medium and (D) a surfactant to form an initial emulsion. The mixture is a discontinuous phase in the aqueous medium (C) of the initial emulsion. The initial emulsion may be formed via the application of shear, e.g. by mixing, shaking, stirring, etc. The discontinuous phase of the initial emulsion is generally present as particles in the aqueous medium (C), or the continuous phase of the initial emulsion. The particles are liquid and may have generally spherical or other shapes, and may have varying sizes based on the components selected and their relative amounts.

The aqueous medium (C) comprises water. The water may be from any source and may optionally be purified, e.g. via distillation, reverse osmosis, etc. The aqueous medium (C) may further comprise one or more additional components other than water, as described below. The aqueous medium (C) typically comprises water in an amount of at least 90, alternatively at least 95, percent by weight based on the total weight of the aqueous medium (C).

The surfactant (D) may be any surfactant capable of emulsifying the various components or improving stability of the initial emulsion. The surfactant (D) may comprise a non-ionic surfactant, an anionic surfactant, or combinations thereof. The amount of the surfactant utilized (D) may vary and is sufficient for forming the initial emulsion, as understood in the art.

Examples of non-ionic surfactants include condensates of ethylene oxide with long chain fatty alcohols or fatty acids; condensates of ethylene oxide with an amine or an amide; condensation products of alkylene oxides (e.g. ethylene and propylene oxide); esters of glycerol; sucrose; sorbitol; fatty acid alkylol amides; sucrose esters; fluoro-surfactants; fatty amine oxides; polyoxyalkylene alkyl ethers, such as polyethylene glycol alkyl ether; polyoxyalkylene sorbitan ethers; polyoxyalkylene alkoxylate esters; polyoxyalkylene alkylphenol ethers; ethylene glycol propylene glycol copolymers and alkylpolysaccharides as described in U.S. Pat. No. 5,035,832, which is incorporated by reference in its entirety. Additional examples of non-ionic surfactants include polymeric surfactants, such as polyvinyl alcohol (PVA) and polyvinylmethylether. Non-ionic surfactants are commercially available from a variety of suppliers.

Examples of anionic surfactants include alkyl sulphates, such as lauryl sulphate; sulfonic acids; alkali metal sulforecinates; sulfonated glyceryl esters of fatty acids, such as sulfonated monoglycerides of coconut oil acids; salts of sulfonated monovalent alcohol esters; amides of amino sulfonic acids; sulfonated products of fatty acid nitriles; sulfonated aromatic hydrocarbons; condensation products of naphthalene sulfonic acids with formaldehyde; sodium octahydroanthracene sulfonate; alkali metal alkyl sulphates; ester sulphates; alkarylsulfonates; alkali metal soaps of higher fatty acids, alkylaryl sulphonates, such as sodium dodecyl benzene sulphonate; long chain fatty alcohol sulphates; olefin sulphates and olefin sulphonates; sulphated monoglycerides; sulphated esters; sulphonated ethoxylated alcohols; sulphosuccinates; alkane sulphonates; phosphate esters; alkyl isethionates; alkyl taurates; and alkyl sarcosinates.

In various embodiments, the surfactant (D) comprises an anionic surfactant selected from a sulfonic acid or a salt thereof. Specific examples of sulfonic acids include, in addition to any described above, alkylsulfonic acids and alkylarylsulfonic acids. Specific alkylarylsulfonic acids include alkylbenzenesulfonic acids, such as hexylbenzenesulfonic acid, octylbenzenesulfonic acid, decylbenzenesulfonic acid, dodecylbenzenesulfonic acid, cetylbenzenesulfonic acid, myristylbenzenesulfonic acid, and alkylnapthylsulfonic acid. The above surfactants may be used individually or in combination. Salts of such sulfonic acids may also be utilized as the surfactant (D) and are known to those of skill in the art.

Some anionic surfactants, such as sulfonic acids (including at least some of the sulfonic acids described above), have catalytic activity for polymerization of the organopolysiloxane (A). The catalytic activity can be suppressed by a neutralizing agent, such as an organic amine, e.g. triethanolamine, or an inorganic base, e.g. sodium hydroxide. As understood in the art, such neutralization of an acid with a base results in a neutralized salt. Such a neutralized salt may be utilized as or along with the surfactant (D), and the neutralized salt may be converted to its acid form, as described below. The neutralized salt from reacting the sulfonic acid with the neutralizing agent is typically a sulphonate.

The method further comprises contacting the organopolysiloxane (A) with (D¹) an organic acid catalyst. The organic acid catalyst (D¹) is the same as or different from the surfactant (D). Contacting the organopolysiloxane (A) organic acid catalyst (D¹) initiates polymerization of the organopolysiloxane (A). Polymerization of the organopolysiloxane (A) takes place in the discontinuous phase of the initial emulsion, i.e., in the organic oil (B) of the mixture, to give (A¹) a high molecular weight organopolysiloxane. The oil-in-water emulsion comprises the high molecular weight organopolysiloxane (A¹) and the organic oil (B) being together in the discontinuous phase, with the aqueous medium (C) being the continuous phase. As in the initial emulsion, the discontinuous phase of the oil-in-water emulsion is present in the form of particles (liquid), which may have varying sizes and shapes, as described below.

The organic acid catalyst (D¹) may be the same as or different from the surfactant (D). In certain embodiments, the organic acid catalyst (D¹) is the same as the surfactant (D). In these embodiments, the surfactant (D), which is the organic acid catalyst (D¹), is present in the initial emulsion to contact the organopolysiloxane (A). Said differently, in these embodiments, contacting the organopolysiloxane (A) with the organic acid catalyst (D¹) occurs upon forming the initial emulsion such that no separate or discrete step of contacting the organopolysiloxane (A) with the organic acid catalyst (D¹) need be carried out. Also, no additional organic acid catalyst (D¹) is needed when the organic acid catalyst (D¹) is the same as the surfactant (D).

When the organic acid catalyst (D¹) is the same as the surfactant (D), the organic acid catalyst (D¹) and the surfactant (D) are typically a sulfonic acid. Suitable sulfonic acids are described above, and are generally selected from alkylsulfonic acids and alkylarylsulfonic acids.

Further, when the organic acid catalyst (D¹) is the same as the surfactant (D), polymerization of the organopolysiloxane (A) may begin upon combining the mixture, the aqueous medium (C) and the surfactant (D) to form the initial emulsion. The components may be combined in any order or manner to form the initial emulsion, optionally in the presence of shear. Polymerization generally continues after formation of the initial emulsion until the high molecular weight organopolysiloxane (A¹) is formed to give the oil-in-water emulsion.

Alternatively, in other embodiments, the organic acid catalyst (D¹) is different from the surfactant (D). For example, it may be desirable to avoid or delay polymerization of the organopolysiloxane (A) until after the formation of the initial emulsion, which can lead to greater control of particle size and viscosity of the high molecular weight organopolysiloxane (A¹) (and its corresponding molecular weight).

In these embodiments, the initial emulsion is formed by combining the mixture, the aqueous medium (C), and the surfactant (D) to the exclusion of the organic acid catalyst (D¹). The organopolysiloxane (A) is subsequently contacted with the organic acid catalyst (D¹).

In certain embodiments when the organic acid catalyst (D¹) is different from the surfactant (D), the organic acid catalyst (D¹) is a sulfonic acid, such as any of the sulfonic acids described above. Alternatively, the organic acid catalyst (D¹) may be a strong acid other than a sulfonic acid, such as sulfuric acid, nitric acid, etc. In these or other embodiments, the surfactant (D) is any of those described above, although the surfactant (D) is typically an anionic surfactant (D).

As introduced above, the neutralized salt formed by reacting a sulfonic acid and a neutralizing agent may be utilized as the surfactant (D). This neutralized salt, in contrast to its acid form, does not catalyze polymerization of the organopolysiloxane (A). As such, in one specific embodiment, the surfactant (D) comprises the neutralized salt. The neutralized salt may be formed at any stage. For example, the neutralized salt may be formed in situ the aqueous medium, in the organic oil (B), in the initial emulsion, etc. Alternatively, the neutralized salt may be first formed or obtained and utilized as a discrete component that is not formed in situ. In these embodiments, the organic acid catalyst (D¹) may be the acid form of the neutralized salt utilized as the surfactant (D). For example, the method may further comprise converting with an acid the surfactant (D) to the organic acid catalyst (D¹). The organic acid catalyst (D¹) is formed upon converting the neutralized salt to its acid form, and the organic acid catalyst (D¹) may upon its formation contact the organopolysiloxane (A) to catalyze polymerization thereof.

When the method further comprises converting with an acid the surfactant to the organic acid catalyst (D¹), the acid may be any acid suitable for such purposes. For example, the acid may be a sulfonic acid (e.g. any of those described above), nitric acid, sulfuric acid, etc.

The high molecular weight organopolysiloxane (A¹) has a greater molecular weight and viscosity than the organopolysiloxane (A). The structure of the high molecular weight organopolysiloxane (A¹) is contingent on the organopolysiloxane (A) utilized, including its content of silicon-bonded hydrolysable or hydroxyl groups, whether the organopolysiloxane (A) includes branching, etc. The high molecular weight organopolysiloxane (A¹) may include some residual functional groups, i.e. silicon-bonded hydrolysable or hydroxyl groups.

In various embodiments, the high molecular weight organopolysiloxane (A¹) is linear. The high molecular weight organopolysiloxane (A¹) typically has a molecular weight of at least 100,000, alternatively at least 150,000, alternatively at least 200,000, g/mol. The molecular weight of the high molecular weight organopolysiloxane (A¹) may reach up to, for example, 500,000 g/mol. The molecular weight of the high molecular weight organopolysiloxane (A¹) is typically measured via gel permeation chromatography techniques (GPC), as understood in the art.

Polymerization of the organopolysiloxane (A) by contacting the organic acid catalyst (D¹) may be carried out at various temperatures, e.g. at room temperature or at a temperature range of from 0 to 30° C.

In certain embodiments, the method further comprises neutralizing any residual amount of the organic acid catalyst (D¹) with a basic compound. The basic compound may be the same as the neutralizing agents described above. Basic compounds for neutralizing organic acids are well known and result in the formation of acid salts. The amount of basic compound utilized is typically in a 1:1 molar ratio with any residual amount of the organic acid catalyst (D¹), although the basic compound may be utilized in varying amounts, e.g. in a molar excess compared to the organic acid catalyst (D¹).

Typically, the high molecular weight organopolysiloxane (A¹) and any residual amount of the organopolysiloxane (A) are present in the oil-in-water emulsion in an amount of from 20 to 60 weight percent based on the total weight of all components utilized to prepare the oil-in-water emulsion. The organic oil (B) is typically present in the oil-in-water emulsion in an amount of from greater than 0 to 20 weight percent based on the total weight of all components utilized to prepare the oil-in-water emulsion. The balance of the oil-in-water emulsion generally comprises the aqueous medium (C), the surfactant (D), the organic acid catalyst (D¹) (if present after any optional neutralization step), and optionally the basic compound. By-products from preparing the high molecular weight organopolysiloxane (A¹) may also be present, e.g. cyclic siloxanes.

The average size of the particles in the oil-in-water emulsion may vary based on the particular embodiment utilized. For example, greater control over particle size may be achieved when the neutralized salt is utilized as the surfactant (D), and the neutralized salt is converted to its acid form to give the organic acid catalyst (D¹) as compared to when the surfactant (D) and the organic acid catalyst (D¹) are the same. Because the particles are generally spherical, the average size of the particles may alternatively be referred to as average particle diameter. Average particle diameters are commonly expressed in DV_{[0.5]} values, which indicates that 50% of the volume of particles in the sample measured have an average particle size below the reported value.

Typically, the average particle size DV_{[0.5]} of the particles in the oil-in-water emulsion is from greater than 0 to 1.5 micrometers (μm). In certain embodiments, the average particle size DV_{[0.5]} may be from greater than 0 to 1,000, alternatively from greater than 100 to 500, nanometers (nm). It various embodiments, it is desirable to minimize average particle size, which provides various advantages in certain end uses of the oil-in-water emulsions. Such values are measured via a dynamic light scattering technique. As understood in the art, the average particle size may vary dependent on the technique utilized to measure the average particle size, and techniques other than dynamic light scattering may be utilized herein. One specific technique is to utilize a particle size analyzer, such as a Mastersizer 2000 particle size analyzer, commercially available from Malvern Instruments Inc. of Westborough, Mass. The particles are typically spherical, but may be irregularly shaped.

The oil-in-water emulsions may further comprise additional components, such as heat stabilizers, flame retardants, UV stabilizers, fungicides, biocides, perfumes, fillers, relaxers, colorants, thickeners, preservatives, or active ingredients such as pharmaceuticals antifoams, freeze thaw stabilizers, inorganic salts to buffer pH, and thickeners. Such components may be added to or present in either the aqueous medium of the oil-in-water emulsion (i.e., the continuous phase) or the particles (i.e., the discontinuous phase).

The oil-in-water emulsions formed in accordance with the method have excellent physical properties and are suitable for use in diverse applications and end uses. For example, the oil-in-water emulsions may be utilized in paints, construction applications, textiles, e.g. fiber treatment, leather lubrication, household care compositions, fabric softening, fabric care in laundry applications, healthcare applications, release agents, water-based coatings, etc. If desired, the high-molecular weight organopolysiloxane (A¹) may be isolated from the oil-in-water emulsion. Alternatively or in addition, by-products prepared along with the high molecular weight organopolysiloxane (A¹) may be removed from the oil-in-water emulsion or separated from the high molecular weight organopolysiloxane (A¹) itself.

The oil-in-water emulsions are particularly well suited for compositions for personal care. Compositions for personal care may alternatively be referred to as cosmetic compositions and include those that are intended to be placed in contact with external portions of the human body (skin, hair, nails, mucosa, etc., also referred to as “keratinous substrates”) or with the teeth and the mucous membranes of the oral cavity with a view exclusively or mainly to cleaning them, perfuming them, changing their appearance, protecting them, keeping them in good condition or modifying odors. In some instances, compositions for personal care also include health care compositions. Cosmetic applications, and in some instances health care applications, include skin care, sun care, hair care, or nail care applications.

The subject invention additionally provides a method of preparing a composition for personal care. This method comprises preparing the oil-in-water emulsion as described above. The method further comprises combining the oil-in-water emulsion with a personal care ingredient. The oil-in-water emulsion and personal care ingredient may be combined in any manner. The personal care ingredient is different from the components of the oil-in-water emulsion. The personal care ingredient may be present along with one or more other components during the step of forming the oil-in-water emulsion such that the composition for personal care is formed in situ. Alternatively, the personal care ingredient may be combined with the oil-in-water emulsion after formation thereof. Typically, the oil-in-water emulsion is first formed and subsequently combined with the personal care ingredient to provide the composition for personal care. Any personal care ingredients or actives from WO2008/045427 and WO2010115782, which are incorporated herein by reference, may be utilized in the composition for personal care.

Personal care ingredients are those components used in personal care or cosmetic applications. A wide review of such components may be found in the CTFA cosmetic component handbook. Exemplary personal care ingredients are described in further detail below. These personal care ingredients may alternative be referred to as cosmetic components, health care components, etc. depending on the typical use thereof. When the personal care ingredient is the cosmetic component, the composition is referred to as a cosmetic composition; when the personal care ingredient is the health care component, the composition is referred to as a health care composition, etc.

Cosmetic components include emollients, waxes, moisturizers, surface active materials such as surfactants or detergents or emulsifiers, thickeners, water phase stabilizing agents, pH controlling agents, preservatives and cosmetic biocides, sebum absorbants or sebum control agents, vegetable or botanical extracts, vitamins, proteins or amino-acids and their derivatives, pigments, colorants, fillers, silicone conditioning agents, cationic conditioning agents, hydrophobic conditioning agents, UV absorbers, sunscreen agents, antidandruff agents, antiperspirant agents, deodorant agents, skin protectants, hair dyes, nail care components, fragrances or perfume, antioxidants, oxidizing agents, reducing agents, propellant gases, and mixtures thereof. Additional components that may be used in the cosmetic compositions include fatty alcohols, color care additives, anticellulites, pearlising agents, chelating agents, film formers, styling agents, ceramides, suspending agents and others.

Health care components include antiacne agents, antibacterial agents, antifungal agents, therapeutic active agents, external analgesics, skin bleaching agents, anti-cancer agents, diuretics, agents for treating gastric and duodenal ulcers, proteolytic enzymes, antihistamine or H1 histamine blockers, sedatives, bronchodilators, diluents, and others. Additional components that may be used in the health care compositions include antibiotics, antiseptics, antibacterial agents, anti-inflammatory agents, astringents, hormones, smoking cessation compositions, cardiovascular agents, antiarrythmic agents, alpha-I blockers, beta blockers, ACE inhibitors, antiaggregants, non-steroidal anti-inflammatory agents such as diclofenac, antipsoriasis agents such as clobetasol propionate, antidermatitis agents, tranquilizer, anticonvulsants, anticoagulant agents, healing factors, cell growth nutrients, peptides, corticosteroidal drugs, antipruritic agents and others.

Cosmetic components may be used in health care compositions, such as waxes, and others; and health care components may be used in cosmetic compositions, such as anti-acne agents, and others.

Examples of emollients include volatile or non-volatile silicone oils; silicone resins, such as polypropylsilsesquioxane and phenyl trimethicone; silicone elastomers, such as dimethicone crosspolymers; alkylmethylsiloxanes, such as C₃₀₋₄₅ alkyl methicone; volatile or non-volatile hydrocarbon compounds, such as squalene, paraffin oils, petrolatum oils and naphthalene oils; hydrogenated or partially hydrogenated polyisobutene; isoeicosane; squalane; isoparaffin; isododecane; isodecane or isohexa-decane; branched C₈-C₁₆ esters; isohexyl neopentanoate; ester oils such as isononyl isononanoate, cetostearyl octanoate, isopropyl myristate, palmitate derivatives, stearates derivatives, isostearyl isostearate and the heptanoates, octanoates, decanoates or ricinoleates of alcohols or of polyalcohols, or mixtures thereof; hydrocarbon oils of plant origin, such as wheatgerm, sunflower, grapeseed, castor, shea, avocado, olive, soybean, sweet almond, palm, rapeseed, cotton seed, hazelnut, macadamia, jojoba, blackcurrant, evening primrose; or triglycerides of caprylic/capric acids; higher fatty acids, such as oleic acid, linoleic acid or linolenic acid, and mixtures thereof.

Example of waxes include hydrocarbon waxes such as beeswax, lanolin wax, rice wax, carnauba wax, candelilla wax, microcrystalline waxes, paraffins, ozokerite, polyethylene waxes, synthetic wax, ceresin, lanolin, lanolin derivatives, cocoa butter, shellac wax, bran wax, capok wax, sugar cane wax, montan wax, whale wax, bayberry wax, silicone waxes (e.g. polymethylsiloxane alkyls, alkoxys and/or esters, C₃₀₋₄₅ alkyldimethylsilyl polypropylsilsesquioxane), and mixtures thereof

Examples of moisturizers include lower molecular weight aliphatic diols such as propylene glycol and butylene glycol; polyols such as glycerine and sorbitol; and polyoxyethylene polymers such as polyethylene glycol 200; hyaluronic acid and its derivatives, and mixtures thereof.

Examples of surface active materials may be anionic, cationic or nonionic, and include organomodified silicones such as dimethicone copolyol; oxyethylenated and/or oxypropylenated ethers of glycerol; oxyethylenated and/or oxypropylenated ethers of fatty alcohols such as ceteareth-30, C₁₂₋₁₅ pareth-7; fatty acid esters of polyethylene glycol such as PEG-50 stearate, PEG-40 monostearate; saccharide esters and ethers, such as sucrose stearate, sucrose cocoate and sorbitan stearate, and mixtures thereof; phosphoric esters and salts thereof, such as DEA oleth-10 phosphate; sulphosuccinates, such as disodium PEG-5 citrate lauryl sulphosuccinate and disodium ricinoleamido MEA sulphosuccinate; alkyl ether sulphates, such as sodium lauryl ether sulphate; isethionates; betaine derivatives; and mixtures thereof.

Further examples of nonionic surfactants include polyoxyethylene alkyl ethers, polyoxyethylene alkylphenol ethers, polyoxyethylene lauryl ethers, polyoxyethylene sorbitan monoleates, polyoxyethylene alkyl esters, polyoxyethylene sorbitan alkyl esters, polyethylene glycol, polypropylene glycol, diethylene glycol, ethoxylated trimethylnonanols, polyoxyalkylene-substituted silicones (rake or ABn types), silicone alkanolamides, silicone esters, silicone glycosides, and mixtures thereof.

Nonionic surfactants include dimethicone copolyols, fatty acid esters of polyols, for instance sorbitol or glyceryl mono-, di-, tri- or sesqui-oleates or stearates, glyceryl or polyethylene glycol laurates; fatty acid esters of polyethylene glycol (polyethylene glycol monostearate or monolaurate); polyoxyethylenated fatty acid esters (stearate or oleate) of sorbitol; polyoxyethylenated alkyl (lauryl, cetyl, stearyl or octyl)ethers.

Anionic surfactants include carboxylates (sodium 2-(2-hydroxyalkyloxy)acetate)), amino acid derivatives (N-acylglutamates, N-acylgly-cinates or acylsarcosinates), alkyl sulfates, alkyl ether sulfates and oxyethylenated derivatives thereof, sulfonates, isethionates and N-acylisethionates, taurates and N-acyl N-methyltaurates, sulfosuccinates, alkylsulfoacetates, phosphates and alkyl phosphates, polypeptides, anionic derivatives of alkyl polyglycoside (acyl-D-galactoside uronate), and fatty acid soaps, and mixtures thereof.

Amphoteric and zwitterionic surfactants include betaines, N-alkylamidobetaines and derivatives thereof, proteins and derivatives thereof, glycine derivatives, sultaines, alkyl polyaminocarboxylates and alkylamphoacetates, and mixtures thereof.

Examples of thickeners include acrylamide copolymers, acrylate copolymers and salts thereof (such as sodium polyacrylate), xanthan gum and derivatives, cellulose gum and cellulose derivatives (such as methylcellulose, methylhydroxypropylcellulose, hydroxypropylcellulose, polypropylhydroxyethylcellulose), starch and starch derivatives (such as hydroxyethylamylose and starch amylase), polyoxyethylene, carbomer, sodium alginate, arabic gum, cassia gum, guar gum and guar gum derivatives, cocamide derivatives, alkyl alcohols, gelatin, PEG-derivatives, saccharides (such as fructose, glucose) and saccharides derivatives (such as PEG-120 methyl glucose diolate), and mixtures thereof.

Examples of water phase stabilizing agents include electrolytes (e.g. alkali metal salts and alkaline earth salts, especially the chloride, borate, citrate, and sulfate salts of sodium, potassium, calcium and magnesium, as well as aluminum chlorohydrate, and polyelectrolytes, especially hyaluronic acid and sodium hyaluronate), polyols (glycerine, propylene glycol, butylene glycol, and sorbitol), alcohols such as ethyl alcohol, and hydrocolloids, and mixtures thereof.

Examples of pH controlling agents include any water soluble acid such as a carboxylic acid or a mineral acid such as hydrochloric acid, sulphuric acid, and phosphoric acid, monocarboxylic acid such as acetic acid and lactic acid, and polycarboxylic acids such as succinic acid, adipic acid, citric acid, and mixtures thereof.

Example of preservatives and cosmetic biocides include paraben derivatives, hydantoin derivatives, chlorhexidine and its derivatives, imidazolidinyl urea, phenoxyethanol, silver derivatives, salicylate derivatives, triclosan, ciclopirox olamine, hexamidine, oxyquinoline and its derivatives, PVP-iodine, zinc salts and derivatives such as zinc pyrithione, and mixtures thereof.

Examples of sebum absorbants or sebum control agents include silica silylate, silica dimethyl silylate, dimethicone/vinyl dimethicone crosspolymer, polymethyl methacrylate, cross-linked methylmethacrylate, aluminum starch octenylsuccinate, and mixtures thereof.

Examples of vegetable or botanical extracts are derived from plants (herbs, roots, flowers, fruits, or seeds) in oil or water soluble form, such as coconut, green tea, white tea, black tea, horsetail, ginkgo biloba, sunflower, wheat germ, seaweed, olive, grape, pomegranate, aloe, apricot kernel, apricot, carrot, tomato, tobacco, bean, potato, actzuki bean, catechu, orange, cucumber, avocado, watermelon, banana, lemon or palm. Examples of herbal extracts include dill, horseradish, oats, neem, beet, broccoli, tea, pumpkin, soybean, barley, walnut, flax, ginseng, poppy, avocado, pea, sesame, and mixtures thereof.

Examples of vitamins include a variety of different organic compounds such as alcohols, acids, sterols, and quinones. They may be classified into two solubility groups: lipid-soluble vitamins and water-soluble vitamins. Lipid-soluble vitamins that have utility in personal care formulations include retinol (vitamin A), ergocalciferol (vitamin D2), cholecalciferol (vitamin D3), phytonadione (vitamin K1), and tocopherol (vitamin E). Water-soluble vitamins that have utility in personal care formulations include ascorbic acid (vitamin C), thiamin (vitamin B1) niacin (nicotinic acid), niacinamide (vitamin B3), riboflavin (vitamin B2), pantothenic acid (vitamin B5), biotin, folic acid, pyridoxine (vitamin B6), and cyanocobalamin (vitamin B12). Additional examples of vitamins include derivatives of vitamins such as retinyl palmitate (vitamin A palmitate), retinyl acetate (vitamin A acetate), retinyl linoleate (vitamin A linoleate), and retinyl propionate (vitamin A propionate), tocopheryl acetate (vitamin E acetate), tocopheryl linoleate (vitamin E linoleate), tocopheryl succinate (vitamin E succinate), tocophereth-5, tocophereth-10, tocophereth-12, tocophereth-18, tocophereth-50 (ethoxylated vitamin E derivatives), PPG-2 tocophereth-5, PPG-5 tocophereth-2, PPG-10 tocophereth-30, PPG-20 tocophereth-50, PPG-30 tocophereth-70, PPG-70 tocophereth-100 (propoxylated and ethoxylated vitamin E derivatives), sodium tocopheryl phosphate, ascorbyl palmitate, ascorbyl dipalmitate, ascorbyl glucoside, ascorbyl tetraisopalmitate, tetrahexadecyl ascorbate, ascorbyl tocopheryl maleate, potassium ascorbyl tocopheryl phosphate, tocopheryl nicotinate, and mixtures thereof.

Examples of proteins or amino-acids and their derivatives include those extracted from wheat, soy, rice, corn, keratin, elastin or silk. Proteins may be in the hydrolyzed form and they may also be quaternized, such as hydrolyzed elastin, hydrolyzed wheat powder, hydrolyzed silk. Examples of protein include enzymes such as hydrolases, cutinases, oxidases, transferases, reductases, hemicellulases, esterases, isomerases, pectinases, lactases, peroxidases, laccases, catalases, and mixtures thereof. Examples of hydrolases include proteases (bacterial, fungal, acid, neutral or alkaline), amylases (alpha or beta), lipases, mannanases, cellulases, collagenases, lisozymes, superoxide dismutase, catalase, and mixtures thereof.

Examples of pigments and colorants include surface treated or untreated iron oxides, surface treated or untreated titanium dioxide, surface treated or untreated mica, silver oxide, silicates, chromium oxides, carotenoids, carbon black, ultramarines, chlorophyllin derivatives and yellow ocher. Examples of organic pigments include aromatic types including azo, indigoid, triphenylmethane, anthraquinone, and xanthine dyes which are designated as D&C and FD&C blues, browns, greens, oranges, reds, yellows, etc., and mixtures thereof. Surface treatments include those treatments based on lecithin, silicone, silanes, fluoro compounds, and mixtures thereof.

Examples of fillers include talc, micas, kaolin, zinc or titanium oxides, calcium or magnesium carbonates, silica, silica silylate, titanium dioxide, glass or ceramic beads, polymethylmethacrylate beads, boron nitride, aluminum silicate, aluminum starch octenylsuccinate, bentonite, magnesium aluminum silicate, nylon, silk powder metal soaps derived from carboxylic acids having 8-22 carbon atoms, non-expanded synthetic polymer powders, expanded powders and powders from natural organic compounds, such as cereal starches, which may or may not be crosslinked, copolymer microspheres, polytrap, silicone resin microbeads, and mixtures thereof. The fillers may be surface treated to modify affinity or compatibility with remaining components.

Examples of silicone conditioning agents include silicone oils such as dimethicone; silicone gums such as dimethiconol; silicone resins such as trimethylsiloxy silicate, polypropyl silsesquioxane; silicone elastomers; alkylmethylsiloxanes; organomodified silicone oils, such as amodimethicone, aminopropyl phenyl trimethicone, phenyl trimethicone, trimethyl pentaphenyl trisiloxane, silicone quaternium-16/glycidoxy dimethicone crosspolymer, silicone quaternium-16; saccharide functional siloxanes; carbinol functional siloxanes; silicone polyethers; siloxane copolymers (divinyldimethicone/dimethicone copolymer); acrylate or acrylic functional siloxanes; and mixtures or emulsions thereof.

Examples of cationic conditioning agents include guar derivatives such as hydroxypropyltrimethylammonium derivative of guar gum; cationic cellulose derivatives, cationic starch derivatives; quaternary nitrogen derivatives of cellulose ethers; homopolymers of dimethyldiallyl ammonium chloride; copolymers of acrylamide and dimethyldiallyl ammonium chloride; homopolymers or copolymers derived from acrylic acid or methacrylic acid which contain cationic nitrogen functional groups attached to the polymer by ester or amide linkages; polymeric quaternary ammonium salts of hydroxyethyl cellulose reacted with a fatty alkyl dimethyl ammonium substituted epoxide; polycondensation products of N,N′-bis-(2,3-epoxypropyl)-piperazine or piperazine-bis-acrylamide and piperazine; and copolymers of vinylpyrrolidone and acrylic acid esters with quaternary nitrogen functionality. Specific materials include the various polyquats Polyquaternium-7, Polyquaternium-8, Polyquaternium-10, Polyquaternium-11, and Polyquaternium-23. Other categories of conditioners include cationic surfactants such as cetyl trimethylammonium chloride, cetyl trimethylammonium bromide, stearyltrimethylammonium chloride, and mixtures thereof. In some instances, the cationic conditioning agent is also hydrophobically modified, such as hydrophobically modified quaternized hydroxyethylcellulose polymers; cationic hydrophobically modified galactomannan ether; and mixtures thereof.

Examples of hydrophobic conditioning agents include guar derivatives; galactomannan gum derivatives; cellulose derivatives; and mixtures thereof.

UV absorbers and sunscreen agents include those which absorb ultraviolet light between about 290-320 nanometers (the UV-B region) and those which absorb ultraviolet light in the range of 320-400 nanometers (the UV-A region).

Some examples of sunscreen agents are aminobenzoic acid, cinoxate, diethanolamine methoxycinnamate, digalloyl trioleate, dioxybenzone, ethyl 4-[bis(Hydroxypropyl)] aminobenzoate, glyceryl aminobenzoate, homosalate, lawsone with dihydroxyacetone, menthyl anthranilate, octocrylene, ethyl hexyl methoxycinnamate, octyl salicylate, oxybenzone, padimate O, phenylbenzimidazole sulfonic acid, red petrolatum, sulisobenzone, titanium dioxide, trolamine salicylate, and mixtures thereof.

Some examples of UV absorbers are acetaminosalol, allatoin PABA, benzalphthalide, benzophenone, benzophenone 1-12, 3-benzylidene camphor, benzylidenecamphor hydrolyzed collagen sulfonamide, benzylidene camphor sulfonic Acid, benzyl salicylate, bornelone, bumetriozole, butyl methoxydibenzoylmethane, butyl PABA, ceria/silica, ceria/silica talc, cinoxate, DEA-methoxycinnamate, dibenzoxazol naphthalene, di-t-butyl hydroxybenzylidene camphor, digalloyl trioleate, diisopropyl methyl cinnamate, dimethyl PABA ethyl cetearyldimonium tosylate, dioctyl butamido triazone, diphenyl carbomethoxy acetoxy naphthopyran, disodium bisethylphenyl tiamminotriazine stilbenedisulfonate, disodium distyrylbiphenyl triaminotriazine stilbenedisulfonate, disodium distyrylbiphenyl disulfonate, drometrizole, drometrizole trisiloxane, ethyl dihydroxypropyl PABA, ethyl diisopropylcinnamate, ethyl methoxycinnamate, ethyl PABA, ethyl urocanate, etrocrylene ferulic acid, glyceryl octanoate dimethoxycinnamate, glyceryl PABA, glycol salicylate, homosalate, isoamyl p-methoxycinnamate, isopropylbenzyl salicylate, isopropyl dibenzolylmethane, isopropyl methoxycinnamate, menthyl anthranilate, menthyl salicylate, 4-methylbenzylidene, camphor, octocrylene, octrizole, octyl dimethyl PABA, ethyl hexyl methoxycinnamate, octyl salicylate, octyl triazone, PABA, PEG-25 PABA, pentyl dimethyl PABA, phenylbenzimidazole sulfonic acid, polyacrylamidomethyl benzylidene camphor, potassium methoxycinnamate, potassium phenylbenzimidazole sulfonate, red petrolatum, sodium phenylbenzimidazole sulfonate, sodium urocanate, TEA-phenylbenzimidazole sulfonate, TEA-salicylate, terephthalylidene dicamphor sulfonic acid, titanium dioxide, triPABA panthenol, urocanic acid, VA/crotonates/methacryloxybenzophenone-1 copolymer, and mixtures thereof.

Examples of antidandruff agents include pyridinethione salts, selenium compounds such as selenium disulfide, and soluble antidandruff agents, and mixtures thereof.

Examples of antiperspirant agents and deodorant agents include aluminum chloride, aluminum zirconium tetrachlorohydrex GLY, aluminum zirconium tetrachlorohydrex PEG, aluminum chlorohydrex, aluminum zirconium tetrachlorohydrex PG, aluminum chlorohydrex PEG, aluminum zirconium trichlorohydrate, aluminum chlorohydrex PG, aluminum zirconium trichlorohydrex GLY, hexachlorophene, benzalkonium chloride, aluminum sesquichlorohydrate, sodium bicarbonate, aluminum sesquichlorohydrex PEG, chlorophyllin-copper complex, triclosan, aluminum zirconium octachlorohydrate, zinc ricinoleate, and mixtures thereof.

Examples of skin protectants include allantoin, aluminum acetate, aluminum hydroxide, aluminum sulfate, calamine, cocoa butter, cod liver oil, colloidal oatmeal, dimethicone, glycerin, kaolin, lanolin, mineral oil, petrolatum, shark liver oil, sodium bicarbonate, talc, witch hazel, zinc acetate, zinc carbonate, zinc oxide, and mixtures thereof.

Examples of hair dyes include 1-acetoxy-2-methylnaphthalene; acid dyes; 5-amino-4-chloro-o-cresol; 5-amino-2,6-dimethoxy-3-hydroxypyridine; 3-amino-2,6-dimethylphenol; 2-amino-5-ethylphenol HCl; 5-amino-4-fluoro-2-methylphenol sulfate; 2-amino-4-hydroxyethylaminoanisole; 2-amino-4-hydroxyethylaminoanisole sulfate; 2-amino-5-nitrophenol; 4-amino-2-nitrophenol; 4-amino-3-nitrophenol; 2-amino-4-nitrophenol sulfate; m-aminophenol HCl; p-aminophenol HCl; m-aminophenol; o-aminophenol; 4,6-bis(2-hydroxyethoxy)-m-phenylenediamine HCl; 2,6-bis(2-hydroxyethoxy)-3,5-pyridinediamine HCl; 2-chloro-6-ethylamino-4-nitrophenol; 2-chloro-5-nitro-N-hydroxyethyl p-phenylenediamine; 2-chloro-p-phenylenediamine; 3,4-diaminobenzoic acid; 4,5-diamino-1-((4-chlorophenyl)methyl)-1H-pyrazole-sulfate; 2,3-diaminodihydropyrazolo pyrazolone dimethosulfonate; 2,6-diaminopyridine; 2,6-diamino-3-((pyridin-3-yl)azo)pyridine; dihydroxyindole; dihydroxyindoline; N,N-dimethyl-p-phenylenediamine; 2,6-dimethyl-p-phenylenediamine; N,N-dimethyl-p-phenylenediamine sulfate; direct dyes; 4-ethoxy-m-phenylenediamine sulfate; 3-ethylamino-p-cresol sulfate; N-ethyl-3-nitro PABA; gluconamidopropyl aminopropyl dimethicone; Haematoxylon brasiletto wood extract; HC dyes; Lawsonia inermis (Henna) extract; hydroxyethyl-3,4-methylenedioxyaniline HCl; hydroxyethyl-2-nitro-p-toluidine; hydroxyethyl-p-phenylenediamine sulfate; 2-hydroxyethyl picramic acid; hydroxypyridinone; hydroxysuccinimidyl C₂₁-C₂₂ isoalkyl acidate; isatin; Isatis tinctoria leaf powder; 2-methoxymethyl-p-phenylenediamine sulfate; 2-methoxy-p-phenylenediamine sulfate; 6-methoxy-2,3-pyridinediamine HCl; 4-methylbenzyl 4,5-diamino pyrazole sulfate; 2,2′-methylenebis 4-aminophenol; 2,2′-methylenebis-4-aminophenol HCl; 3,4-methylenedioxyaniline; 2-methylresorcinol; methylrosanilinium chloride; 1,5-naphthalenediol; 1,7-naphthalenediol; 3-nitro-p-Cresol; 2-nitro-5-glyceryl methylaniline; 4-nitroguaiacol; 3-nitro-p-hydroxyethylaminophenol; 2-nitro-N-hydroxyethyl-p-anisidine; nitrophenol; 4-nitrophenyl aminoethylurea; 4-nitro-o-phenylenediamine dihydrochloride; 2-nitro-p-phenylenediamine dihydrochloride; 4-nitro-o-phenylenediamine HCl; 4-nitro-m-phenylenediamine; 4-nitro-o-phenylenediamine; 2-nitro-p-phenylenediamine; 4-nitro-m-phenylenediamine sulfate; 4-nitro-o-phenylenediamine sulfate; 2-nitro-p-phenylenediamine sulfate; 6-nitro-2,5-pyridinediamine; 6-nitro-o-toluidine; PEG-3 2,2′-di-p-phenylenediamine; p-phenylenediamine HCl; p-phenylenediamine sulfate; phenyl methyl pyrazolone; N-phenyl-p-phenylenediamine HCl; pigment blue 15:1; pigment violet 23; pigment yellow 13; pyrocatechol; pyrogallol; resorcinol; sodium picramate; sodium sulfanilate; solvent yellow 85; solvent yellow 172; tetraaminopyrimidine sulfate; tetrabromophenol blue; 2,5,6-triamino-4-pyrimidinol sulfate; 1,2,4-trihydroxybenzene.

Example of nail care components include butyl acetate; ethyl acetate; nitrocellulose; acetyl tributyl citrate; isopropyl alcohol; adipic acid/neopentyl glycol/trimelitic anhydride copolymer; stearalkonium bentonite; acrylates copolymer; calcium pantothenate; Cetraria islandica extract; Chondrus crispus; styrene/acrylates copolymer; trimethylpentanediyl dibenzoate-1; polyvinyl butyral; N-butyl alcohol; propylene glycol; butylene glycol; mica; silica; tin oxide; calcium borosilicate; synthetic fluorphlogopite; polyethylene terephtalate; sorbitan laurate derivatives; talc; jojoba extract; diamond powder; isobutylphenoxy epoxy resin; silk powder; and mixtures thereof.

Examples of fragrances or perfume include hexyl cinnamic aldehyde; anisaldehyde; methyl-2-n-hexyl-3-oxo-cyclopentane carboxylate; dodecalactone gamma; methylphenylcarbinyl acetate; 4-acetyl-6-tert-butyl-1,1-dimethyl indane; patchouli; olibanum resinoid; labdanum; vetivert; copaiba balsam; fir balsam; 4-(4-hydroxy-4-methyl pentyl)-3-cyclohexene-1-carboxaldehyde; methyl anthranilate; geraniol; geranyl acetate; linalool; citronellol; terpinyl acetate; benzyl salicylate; 2-methyl-3-(p-isopropylphenyl)-propanal; phenoxyethyl isobutyrate; cedryl acetal; aubepine; musk fragrances; macrocyclic ketones; macrolactone musk fragrances; ethylene brassylate; and mixtures thereof. Further perfume components are described in detail in standard textbook references such as Perfume and Flavour Chemicals, 1969, S. Arctander, Montclair, N.J.

Examples of antioxidants are acetyl cysteine, arbutin, ascorbic acid, ascorbic acid polypeptide, ascorbyl dipalmitate, ascorbyl methylsilanol pectinate, ascorbyl palmitate, ascorbyl stearate, BHA, p-hydroxyanisole, BHT, t-butyl hydroquinone, caffeic acid, Camellia sinensis oil, chitosan ascorbate, chitosan glycolate, chitosan salicylate, chlorogenic acids, cysteine, cysteine HCl, decyl mercaptomethylimidazole, erythorbic acid, diamylhydroquinone, di-t-butylhydroquinone, dicetyl thiodipropionate, dicyclopentadiene/t-butylcresol copolymer, digalloyl trioleate, dilauryl thiodipropionate, dimyristyl thiodipropionate, dioleyl tocopheryl methylsilanol, isoquercitrin, diosmine, disodium ascorbyl sulfate, disodium rutinyl disulfate, distearyl thiodipropionate, ditridecyl thiodipropionate, dodecyl gallate, ethyl ferulate, ferulic acid, hydroquinone, hydroxylamine HCl, hydroxylamine sulfate, isooctyl thioglycolate, kojic acid, madecassicoside, magnesium ascorbate, magnesium ascorbyl phosphate, melatonin, methoxy-PEG-7 rutinyl succinate, methylene di-t-butylcresol, methylsilanol ascorbate, nordihydroguaiaretic acid, octyl gallate, phenylthioglycolic acid, phloroglucinol, potassium ascorbyl tocopheryl phosphate, thiodiglycolamide, potassium sulfite, propyl gallate, rosmarinic acid, rutin, sodium ascorbate, sodium ascorbyl/cholesteryl phosphate, sodium bisulfite, sodium erythorbate, sodium metabisulfide, sodium sulfite, sodium thioglycolate, sorbityl furfural, tea tree (Melaleuca aftemifolia) oil, tocopheryl acetate, tetrahexyldecyl ascorbate, tetrahydrodiferuloylmethane, tocopheryl linoleate/oleate, thiodiglycol, tocopheryl succinate, thiodiglycolic acid, thioglycolic acid, thiolactic acid, thiosalicylic acid, thiotaurine, retinol, tocophereth-5, tocophereth-10, tocophereth-12, tocophereth-18, tocophereth-50, tocopherol, tocophersolan, tocopheryl linoleate, tocopheryl nicotinate, tocoquinone, o-tolyl biguanide, tris(nonylphenyl) phosphite, ubiquinone, zinc dibutyldithiocarbamate, and mixtures thereof.

Examples of oxidizing agents are ammonium persulfate, calcium peroxide, hydrogen peroxide, magnesium peroxide, melamine peroxide, potassium bromate, potassium caroate, potassium chlorate, potassium persulfate, sodium bromate, sodium carbonate peroxide, sodium chlorate, sodium iodate, sodium perborate, sodium persulfate, strontium dioxide, strontium peroxide, urea peroxide, zinc peroxide, and mixtures thereof.

Examples of reducing agents are ammonium bisufite, ammonium sulfite, ammonium thioglycolate, ammonium thiolactate, cystemaine HCl, cystein, cysteine HCl, ethanolamine thioglycolate, glutathione, glyceryl thioglycolate, glyceryl thioproprionate, hydroquinone, p-hydroxyanisole, isooctyl thioglycolate, magnesium thioglycolate, mercaptopropionic acid, potassium metabisulfite, potassium sulfite, potassium thioglycolate, sodium bisulfite, sodium hydrosulfite, sodium hydroxymethane sulfonate, sodium metabisulfite, sodium sulfite, sodium thioglycolate, strontium thioglycolate, superoxide dismutase, thioglycerin, thioglycolic acid, thiolactic acid, thiosalicylic acid, zinc formaldehyde sulfoxylate, and mixtures thereof.

Examples of propellant gases include carbon dioxide, nitrogen, nitrous oxide, volatile hydrocarbons such as butane, isobutane, or propane, and chlorinated or fluorinated hydrocarbons such as dichlorodifluoromethane and dichlorotetrafluoroethane or dimethylether; and mixtures thereof.

Examples of antiacne agents include salicylic acid, sulfur benzoyl, peroxide, tretinoin, and mixtures thereof.

Examples of antibacterial agents include chlorohexadiene gluconate, alcohol, benzalkonium chloride, benzethonium chloride, hydrogen peroxide, methylbenzethonium chloride, phenol, poloxamer 188, povidone-iodine, and mixtures thereof.

Examples of antifungal agents include miconazole nitrate, calcium undecylenate, undecylenic acid, zinc undecylenate, and mixtures thereof.

Examples of therapeutic active agents include penicillins, cephalosporins, tetracyclines, macrolides, epinephrine, amphetamines, aspirin, acetominophen, barbiturates, catecholamines, benzodiazepine, thiopental, codeine, morphine, procaine, lidocaine, benzocaine, sulphonamides, ticonazole, perbuterol, furosamide, prazosin, hormones, prostaglandins, carbenicillin, salbutamol, haloperidol, suramin, indomethicane, diclofenac, glafenine, dipyridamole, theophylline, hydrocortisone, steroids, scopolamine, and mixtures thereof.

Examples of external analgesics are benzyl alcohol, capsicum oleoresin (Capsicum frutescens oleoresin), methyl salicylate, camphor, phenol, capsaicin, juniper tar (Juniperus oxycedrus tar), phenolate sodium (sodium phenoxide), capsicum (Capsicum frutescens), menthol, resorcinol, methyl nicotinate, turpentine oil (turpentine), and mixtures thereof.

An example of a skin bleaching agent is hydroquinone.

Examples of anti-cancer agents include alkylating agents (such as busulfan, fluorodopan), antimitotic agents (such as colchicine, rhizoxin), topoisomerase I inhibitors (such as camptothecin and its derivatives), topoisomerase II inhibitors (such as menogaril, amonafide), RNA/DNA or DNA anti-metabolites (such as acivicin, guuanazole), plant alkaloids and terpenoids, antineoplastics, some plant-derived compounds (such as podophyllotoxin, vinca alkaloids), and mixtures thereof.

Examples of diuretics include loop diuretics (such as bumetanide, furosemide), thiazide diuretics (such as chlorothiazide, hydroflumethiazide), potassium-sparing diuretics (such as amioloride, spironolactone), carbonic anhydrase inhibitors (such as acetazolamide), osmotic diuretics (such as mannitol), and mixtures thereof.

Examples of agents for treating gastric and duodenal ulcers include proton pump inhibitor (such as lansoprazole, omeprazole), acid blockers or H2 histamine blockers (such as cimetidine, ranitidine), bismuth, sucralfate, and mixtures thereof.

Examples of proteolytic enzymes include nattokinase, serratiopeptidase, bromelain, papain, and mixtures thereof.

Examples of antihistamine or H1 histamine blockers include brompheniramine, clemastine, cetirizine, loratadine, fexofenadine, and mixtures thereof.

Examples of sedatives include barbiturates (such as phenobarbitol), benzodiazepines (such as lorazepam), herbal sedatives, benzodiazepine-like drugs (such as zolpidem, zopiclone), and mixtures thereof.

Examples of bronchodilators include short-acting β2-agonists and long-acting β2-agonists, anticholinergics, and mixtures thereof.

The formulations of the present invention also include diluents. Such diluents are often necessary to decrease the viscosity of the formulation sufficiently for application.

Examples of diluents include silicon containing diluents such as hexamethyldisiloxane, octamethyltrisiloxane, and other short chain linear siloxanes such as octamethyltrisiloxane, decamethyltetrasiloxane, dodecamethylpentasiloxane, tetradecamethylhexasiloxane, hexadeamethylheptasiloxane, heptamethyl-3-{(trimethylsilyl)oxy)}trisiloxane, cyclic siloxanes such as hexamethylcyclotrisiloxane, octamethylcyclotetrasiloxane, decamethylcyclopentasiloxane, dodecamethylcyclohexasiloxane; organic diluents such as butyl acetate, alkanes, alcohols, ketones, esters, ethers, glycols, glycol ethers, hydrofluorocarbons or any other material which can dilute the formulation without adversely affecting any of the component materials of the cosmetic composition. Hydrocarbons include isododecane, isohexadecane, Isopar L (C₁₁-C₁₃), Isopar H (C₁₁-C₁₂), hydrogentated polydecene. Ethers and esters include isodecyl neopentanoate, neopentylglycol heptanoate, glycol distearate, dicaprylyl carbonate, diethylhexyl carbonate, propylene glycol n butyl ether, ethyl-3 ethoxypropionate, propylene glycol methyl ether acetate, tridecyl neopentanoate, propylene glycol methylether acetate (PGMEA), propylene glycol methylether (PGME), octyldodecyl neopentanoate, diisobutyl adipate, diisopropyl adipate, propylene glycol dicaprylate/dicaprate, and octyl palmitate. Additional organic diluents include fats, oils, fatty acids, and fatty alcohols.

The amount of the oil-in-water emulsion in the compositions described above may vary from 0.1 to 95, alternatively from 0.2 to 50, alternatively from 0.5 to 25, weight percent relative to the total weight of the composition. The personal care ingredient is present in an amount of from 0.01 to 99.99 weight percent relative to the total weight of the composition. Combinations of different personal care ingredients may be utilized.

The compositions may be in the form of a cream, a gel, a powder (free flowing powder or pressed), a paste, a solid, freely pourable liquid, or an aerosol. The compositions may be in the form of monophasic systems; biphasic or alternate multi phasic systems; emulsions, e.g. oil-in-water, water-in-oil, silicone-in-water, water-in-silicone; multiple emulsions, e.g. oil-in-water-in-oil, polyol-in-silicone-in-water, oil-in-water-in-silicone.

Skin care compositions include shower gels; soaps; hydrogels; creams; lotions and balms; antiperspirants; deodorants, such as sticks, soft solid, roll on, aerosol, and pumpsprays; skin creams; skin care lotions; moisturizers; facial treatments, such as wrinkle control or diminishment treatments; exfoliates; body and facial cleansers; bath oils; perfumes; colognes; sachets; sunscreens; mousses; patches; pre-shave and after-shave lotions; shaving soaps; shaving lathers; depilatories; make-ups; color cosmetics; foundations; concealers; blushes; lipsticks; eyeliners; mascaras; oil removers; color cosmetic removers, powders, and kits thereof.

Hair care compositions include shampoos, rinse-off conditioners, leave-in conditioners and styling aids, gels, sprays, pomades, mousses, waxes, hair colorants, hair relaxants, hair straighteners, permanents, and kits thereof.

Nail care compositions include color coats, base coats, cuticle coats, nail hardeners, and kits thereof.

Health care compositions may be in the form of ointments, creams, gels, mousses, pastes, patches, spray on bandages, foams and/or aerosols or the like, medicament creams, pastes or sprays including anti-acne, dental hygienic, antibiotic, healing promotive, which may be preventative and/or therapeutic medicaments, and kits thereof.

The compositions may be used by standard methods, such as applying them to the human or animal body, e.g. skin or hair, using applicators, brushes, applying by hand, pouring them and/or optionally rubbing or massaging the composition onto or into the body.

The cosmetic compositions are applied topically to the desired area of the skin or hair in an amount sufficient to provide a satisfactory cleansing or conditioning of the skin or hair. The compositions may be diluted with water prior to, during, or after topical application, and then subsequently rinsed or wiped off of the applied surface, for example rinsed off of the applied surface using water or a water-insoluble substrate in combination with water.

The compositions may be used on hair in a conventional manner. An effective amount of the composition for washing or conditioning hair is applied to the hair, with the effective amount typically ranging from 1 to 50 grams. Application to the hair typically includes working the composition through the hair such that most or all of the hair is contacted with the cosmetic composition. These steps can be repeated as many times as desired to achieve the desired benefit.

Benefits obtained from using the cosmetic compositions on hair include one or more of the following benefits: color retention, improvement in coloration process, hair conditioning, softness, detangling ease, silicone deposition, anti-static, anti-frizz, lubricity, shine, strengthening, viscosity, tactile, wet combing, dry combing, straightening, heat protection, styling, and curl retention.

The compositions may be used on skin in a conventional manner. An effective amount of the composition for the purpose is applied to the skin, with the effective amount typically ranging from 1 to 3 mg/cm². Application to the skin typically includes working the composition into the skin as many times as desired to achieve the desired benefit.

Benefits obtained from using the cosmetic compositions on skin include one or more of the following benefits: stability in various formulations (o/w, w/o, anhydrous), utility as an emulsifier, level of hydrophobicity, organic compatibility, substantivity/durability, wash off resistance, interactions with sebum, performance with pigments, pH stability, skin softness, suppleness, moisturization, skin feel, long lasting, long wear, long lasting color uniformity, color enhancement, foam generation, optical effects (soft focus), and stabilization of actives.

The composition may be used to care for keratinous substrates, to cleanse, to condition, to refresh, to make up, to remove make up, or to fix hair.

It is to be understood that the appended claims are not limited to express and particular compounds, compositions, or methods described in the detailed description, which may vary between particular embodiments which fall within the scope of the appended claims. With respect to any Markush groups relied upon herein for describing particular features or aspects of various embodiments, different, special, and/or unexpected results may be obtained from each member of the respective Markush group independent from all other Markush members. Each member of a Markush group may be relied upon individually and or in combination and provides adequate support for specific embodiments within the scope of the appended claims.

Further, any ranges and subranges relied upon in describing various embodiments of the present invention independently and collectively fall within the scope of the appended claims, and are understood to describe and contemplate all ranges including whole and/or fractional values therein, even if such values are not expressly written herein. One of skill in the art readily recognizes that the enumerated ranges and subranges sufficiently describe and enable various embodiments of the present invention, and such ranges and subranges may be further delineated into relevant halves, thirds, quarters, fifths, and so on. As just one example, a range “of from 0.1 to 0.9” may be further delineated into a lower third, i.e., from 0.1 to 0.3, a middle third, i.e., from 0.4 to 0.6, and an upper third, i.e., from 0.7 to 0.9, which individually and collectively are within the scope of the appended claims, and may be relied upon individually and/or collectively and provide adequate support for specific embodiments within the scope of the appended claims. In addition, with respect to the language which defines or modifies a range, such as “at least,” “greater than,” “less than,” “no more than,” and the like, it is to be understood that such language includes subranges and/or an upper or lower limit. As another example, a range of “at least 10” inherently includes a subrange of from at least 10 to 35, a subrange of from at least 10 to 25, a subrange of from 25 to 35, and so on, and each subrange may be relied upon individually and/or collectively and provides adequate support for specific embodiments within the scope of the appended claims. Finally, an individual number within a disclosed range may be relied upon and provides adequate support for specific embodiments within the scope of the appended claims. For example, a range “of from 1 to 9” includes various individual integers, such as 3, as well as individual numbers including a decimal point (or fraction), such as 4.1, which may be relied upon and provide adequate support for specific embodiments within the scope of the appended claims.

### EXAMPLES

The following examples are intended to illustrate the invention and are not to be viewed in any way as limiting to the scope of the invention.

Various oil-in-water emulsions are prepared in accordance with the inventive method. The tables below detail the relative amounts of types of each component in each oil-in-water emulsion formed.

In each of the examples below, (A) an organopolysiloxane including at least two silicon-bonded hydroxyl groups and (B) an organic oil to give a mixture. In these examples, the ratio of (A):(B) is 4:1 based on weight. Separately, (E) a basic compound and (D) a surfactant are combined with water to give an aqueous medium. The mixture is disposed in the aqueous medium at a 50:50 weight ratio, and this combination is sheared via a high shear device (an ULTRA-TURRAX® device commercially available from IKA® Works, Inc. of Wilmington, N.C.) to provide an initial emulsion. The initial emulsion is subjected to an extra shearing step with a high pressure homogenizer (Rannie, commercially available from SPX Corporation of Delavan, Wis.). Then, (D¹) an organic acid catalyst and additional water is combined with the initial emulsion until pH of the pre-emulsion reaches 1. The organopolysiloxane (A) contacts the organic acid catalyst (D¹) to polymerize the organopolysiloxane (A). Polymerization of the organopolysiloxane proceeds for about 24 hours. Temperature is maintained at 21° C. for 10 minutes and subsequently reduced to 10° C. over a 4 hour period. pH is then increased via further addition of the basic compound (E) until a pH of 7 is achieved. The resulting oil-in-water emulsion comprises (A¹) a high molecular weight organopolysiloxane.

### Example 1

Table 1 below identifies the components and relative amounts utilized in the oil-in-water emulsion formed based on the procedure described above. The amounts are based on the total weight of the oil-in-water emulsion.

TABLE 1

| Component | % by weight |
|---|---|
| Organopolysiloxane | 40.0 |
| Organic oil 1 | 7.1 |
| Basic compound | 6.3 |
| Surfactant 1 | 2.6 |
| Organic acid catalyst precursor | 9.6 |
| Water | 34.4 |

Organopolysiloxane is a hydroxyl-terminated polydimethylsiloxane having a molecular weight of from 5,000 to 9,000 g/mol.

Organic oil 1 is mineral oil.

Basic compound is triethanolamine.

Surfactant 1 is alkylbenzenesulfonic acid.

Organic acid catalyst is the alkylbenzenesulfonic acid, once acidified via the organic acid catalyst precursor, which is sulfuric acid (10%).

The mean particle size of the oil-in-water emulsion is 0.14 micron. Mean particle size is measured via a Malvern MasterSizer 2000. Molecular weight of the high molecular weight organopolysiloxane (A¹) is 275,100 g/mol, as measured via gel permeation chromatography techniques (GPC).

### Example 2

Table 2 below identifies the components and relative amounts utilized in the oil-in-water emulsion formed based on the procedure described above. The amounts are based on the total weight of the oil-in-water emulsion.

TABLE 2

| Component | % by weight |
|---|---|
| Organopolysiloxane | 55.0 |
| Organic oil 2 | 5 |
| Surfactant 1/Organic acid catalyst | 1.2 |
| Basic compound | 0.65 |
| Water | 38.15 |

The surfactant 1 and organic acid catalyst are the same and are alkylbenzenesulfonic acid.

Organic oil 2 is a hydrogenated polydecene, commercially available under the tradename Silkflo® 366 NF from INEOS USA LLC of League City, Tex.

The mean particle size of the oil-in-water emulsion is 0.68 micron after 21 hours of polymerization, as measured via a MasterSizer 2000, commercially available from Malvern Instruments Ltd. of Malvern, UK.

### Example 3

Table 3 below identifies the components and relative amounts utilized in the oil-in-water emulsion formed based on the procedure described above. The amounts are based on the total weight of the oil-in-water emulsion.

TABLE 3

| Component | % by weight |
|---|---|
| Organopolysiloxane | 55.0 |
| Organic oil 3 | 5 |
| Surfactant 1/Organic acid catalyst | 1.2 |
| Basic compound | 0.65 |
| Water | 38.15 |

Organic oil 3 is olive oil.

The mean particle size of the oil-in-water emulsion is 1.2 micron after 21 hours of polymerization, as measured via a Malvern MasterSizer 2000.

### Examples 4-7

Examples 4-7 highlight an additional dilution phase prior to contacting the organopolysiloxane (A) with the organic acid catalyst (D¹).

Table 4 below highlights the types and relative amount of each component in the initial emulsions of Examples 4-7. The amounts are based on the total weight of the particular oil-in-water emulsion rather than the initial emulsion. Because there is a subsequent dilution step in Examples 4-7, the total % by weight in Table 4 is 74.88% of the total weight of the oil-in-water emulsion.

TABLE 4

| | Example | Example | Example | Example |
|---|---|---|---|---|
| | 4 (% by | 5 (% by | 6 (% by | 7 (% by |
| Component | weight) | weight) | weight) | weight) |
| Organopolysiloxane | 40 | 40 | 40 | 40 |
| Basic compound | 2.24 | 2.24 | 2.24 | 2.24 |
| Surfactant 2 | 2.64 | 2.64 | 2.64 | 2.64 |
| Initial Water | 20 | 20 | 20 | 20 |
| Organic oil 1 | — | — | — | 10 |
| Organic oil 3 | — | 10 | — | — |
| Organic oil 4 | — | — | 10 | — |
| Organic oil 5 | 10 | — | — | — |

Surfactant 2 is dodecylbenzenesulfonate.

Organic oil 4 is avocado oil.

Organic oil 5 is grape seed oil.

After forming each of the initial emulsions illustrated in Table 4 above, an additional amount of water is added, along with an organic acid catalyst precursor to convert the surfactant 2 to its acidified form, and an additional amount of the basic compound to neutralize any acidity. Further, a personal care ingredient is included in the oil-in-water emulsions of Examples 4-7.

Table 5 below highlights the types and relative amount of each component combined with the initial emulsions of Table 4 to give the respective oil-in-water emulsions of Example 4-7. The amounts are based on the total weight of the particular oil-in-water emulsion rather than the initial emulsion. Because there is an initial emulsion formed in Examples 4-7, the total % by weight in Table 5 is 25.12% of the total weight of the oil-in-water emulsion.

TABLE 5

| | Example | Example | Example | Example |
|---|---|---|---|---|
| | 4 (% by | 5 (% by | 6 (% by | 7 (% by |
| Component | weight) | weight) | weight) | weight) |
| Water | 18.93 | 18.93 | 18.93 | 18.93 |
| Basic compound | 2.7 | 2.7 | 2.7 | 2.7 |
| Organic acid | 3.4 | 3.4 | 3.4 | 3.4 |
| catalyst precursor | | | | |
| Personal Care | 0.09 | 0.09 | 0.09 | 0.09 |
| ingredient | | | | |

Personal care ingredient is an isothiazolinone, commercially available under the tradename Kathon™ from the Dow Chemical Company of Midland, Mich.

Additional embodiments of this disclosure are as follows:
1. A method of preparing an oil-in-water emulsion, said method comprising:

combining (A) an organopolysiloxane including at least two silicon-bonded hydrolysable or hydroxyl groups and (B) an organic oil to give a mixture;

combining the mixture, (C) an aqueous medium and (D) a surfactant to form an initial emulsion, wherein the mixture is a discontinuous phase in the aqueous medium (C) of the initial emulsion; and

contacting the organopolysiloxane (A) with (D¹) an organic acid catalyst, which is the same as or different from the surfactant (D), to polymerize the organopolysiloxane (A) in the discontinuous phase of the initial emulsion to give (A¹) a high molecular weight organopolysiloxane, thereby preparing the oil-in-water emulsion.
2. The method of embodiment 1, further comprising neutralizing any residual amount of the organic acid catalyst (D¹) with (E) a basic compound.
3. The method of embodiment 1 or 2, wherein the organopolysiloxane (A): (i) is linear; (ii) includes the at least two silicon-bonded hydrolysable or hydroxyl groups at opposite terminal locations; or (iii) both (i) and (ii).
4. The method of any one of the preceding embodiments, wherein the organopolysiloxane (A) has the formula: (R_{3-w}X_{w}SiO_{1/2})ₐ(R₂₋ₓXₓSiO_{2/2})_{b}(R_{1-y}X_{y}SiO_{3/2})_{c}(SiO_{4/2})_{d}, wherein each R is independently a substituted or unsubstituted hydrocarbyl group, each X is independently selected from R and a hydrolysable or hydroxyl group, w is an integer from 0 to 3, x is an integer from 0 to 2, and y is 0 or 1, with the proviso that (w+x+y)≥2 and (a+b+c+d)=1.
5. The method of any one of the preceding embodiments, wherein the organic acid catalyst (D¹) comprises an alkylsulfonic acid or an alkylarylsulfonic acid.
6. The method of embodiment 5, wherein the organic acid catalyst (D¹) is an alkylbenzenesulfonic acid.
7. The method of any one of the preceding embodiments, wherein the surfactant (D) is different from the organic acid catalyst (D¹).
8. The method of embodiment 7, wherein the surfactant (D) is a neutralized salt of the organic acid catalyst (D¹), and wherein the method further comprises converting with an acid the surfactant (D) to the organic acid catalyst (D¹) to contact the organopolysiloxane (A).
9. The method of any one of embodiments 1-6, wherein the surfactant (D) is the organic acid catalyst (D¹) such that the organic acid catalyst (D¹) is present in the initial emulsion to contact the organopolysiloxane (A).
10. The method of any one of the preceding embodiments, wherein the high molecular weight organopolysiloxane (A¹) is present along with the organic oil (B) in the form of particles dispersed in the aqueous medium (C) in the oil-in-water emulsion, and wherein the particles have an average largest dimension of from greater than 0 to 1.5 micrometers (μm).
11. The method of any one of the preceding embodiments, wherein the organopolysiloxane (A) is utilized in an amount of from greater than 20 to 60 weight percent based on the total weight of all components utilized to prepare the oil-in-water emulsion and wherein the organic oil (B) is utilized in an amount of from greater than 0 to 20 weight percent based on the total weight of all components utilized to prepare the oil-in-water emulsion.
12. The method of any one of the preceding embodiments, wherein the surfactant (D) is anionic.
13. The method of any one of the preceding embodiments, wherein the organic oil (B) is selected from a paraffin oil, a hydrogenated polyolefin oil, a fatty acid ester oil, a vegetable oil, or a combination thereof.
14. The method of any one of the preceding embodiments, wherein the organopolysiloxane (A) has a dynamic viscosity of from 0.02 to 150 Pa·s at 25° C.
15. The method of any one of the preceding embodiments, wherein the high molecular weight organopolysiloxane (A¹) has a dynamic viscosity of from 500 to 10,000 Pa·s at 25° C.
16. An oil-in-water emulsion formed in accordance with the method of embodiment 1, alternatively of any one of the preceding embodiments.
17. A composition for personal care comprising the oil-in-water emulsion of embodiment 16 and at least one personal care ingredient.
18. A method of preparing a composition for personal care, said method comprising:

combining (A) an organopolysiloxane including at least two silicon-bonded hydrolysable or hydroxyl groups and (B) an organic oil to give a mixture;

combining the mixture, (C) an aqueous medium and (D) a surfactant to form an initial emulsion, wherein the mixture is a discontinuous phase in the aqueous medium (C);

contacting the organopolysiloxane (A) with an organic acid catalyst (D¹), which is the same as or different from the surfactant (D), to polymerize the organopolysiloxane (A) in the discontinuous phase of the initial emulsion to give a high molecular weight organopolysiloxane (A¹), thereby preparing an oil-in-water emulsion; and

combining the oil-in-water emulsion with at least one personal care ingredient.

The invention has been described herein in an illustrative manner, and it is to be understood that the terminology which has been used is intended to be in the nature of words of description rather than of limitation. Many modifications and variations of the present invention are possible in light of the above teachings. The invention may be practiced otherwise than as specifically described within the scope of the appended claims. The subject matter of all combinations of independent and dependent claims, both single and multiple dependent, is herein expressly contemplated.

1. A method of preparing an oil-in-water emulsion, said method comprising:
combining (A) an organopolysiloxane including at least two silicon-bonded hydrolysable or hydroxyl groups and (B) an organic oil to give a mixture;
combining the mixture, (C) an aqueous medium and (D) a surfactant to form an initial emulsion, wherein the mixture is a discontinuous phase in the aqueous medium (C) of the initial emulsion; and
contacting the organopolysiloxane (A) with (D¹) an organic acid catalyst, which is the same as or different from the surfactant (D), to polymerize the organopolysiloxane (A) in the discontinuous phase of the initial emulsion to give (A¹) a high molecular weight organopolysiloxane, thereby preparing the oil-in-water emulsion.

2. The method of claim 1, further comprising neutralizing any residual amount of the organic acid catalyst (D¹) with (E) a basic compound.

3. The method of claim 1, wherein the organopolysiloxane (A): (i) is linear; (ii) includes the at least two silicon-bonded hydrolysable or hydroxyl groups at opposite terminal locations; or (iii) both (i) and (ii).

4. The method of claim 1, wherein the organopolysiloxane (A) has the formula: (R_{3-w}X_{w}SiO_{1/2})ₐ(R₂₋ₓXₓSiO_{2/2})_{b}(R_{1-y}X_{y}SiO_{3/2})_{c}(SiO_{4/2})_{d}, wherein each R is independently a substituted or unsubstituted hydrocarbyl group, each X is independently selected from R and a hydrolysable or hydroxyl group, w is an integer from 0 to 3, x is an integer from 0 to 2, and y is 0 or 1, with the proviso that (w+x+y)≥2 and (a+b+c+d)=1.

5. The method of claim 1, wherein the organic acid catalyst (D¹) comprises an alkylsulfonic acid or an alkylarylsulfonic acid.

6. The method of claim 5 wherein the organic acid catalyst (D¹) is an alkylbenzenesulfonic acid.

7. The method of claim 1, wherein the surfactant (D) is different from the organic acid catalyst (D¹).

8. The method of claim 7, wherein the surfactant (D) is a neutralized salt of the organic acid catalyst (D¹), and wherein the method further comprises converting with an acid the surfactant (D) to the organic acid catalyst (D¹) to contact the organopolysiloxane (A).

9. The method of claim 1, wherein the surfactant (D) is the organic acid catalyst (D¹) such that the organic acid catalyst (D¹) is present in the initial emulsion to contact the organopolysiloxane (A).

10. The method of claim 1, wherein the high molecular weight organopolysiloxane (A¹) is present along with the organic oil (B) in the form of particles dispersed in the aqueous medium (C) in the oil-in-water emulsion, and wherein the particles have an average largest dimension of from greater than 0 to 1.5 micrometers (μm).

11. The method of claim 1, wherein the organopolysiloxane (A) is utilized in an amount of from greater than 20 to 60 weight percent based on the total weight of all components utilized to prepare the oil-in-water emulsion and wherein the organic oil (B) is utilized in an amount of from greater than 0 to 20 weight percent based on the total weight of all components utilized to prepare the oil-in-water emulsion.

12. The method of claim 1, wherein the surfactant (D) is anionic.

13. The method of claim 1, wherein the organic oil (B) is selected from a paraffin oil, a hydrogenated polyolefin oil, a fatty acid ester oil, a vegetable oil, or a combination thereof.

14. The method of claim 1, wherein the organopolysiloxane (A) has a dynamic viscosity of from 0.02 to 150 Pa·s at 25° C.

15. The method of claim 1, wherein the high molecular weight organopolysiloxane (A¹) has a dynamic viscosity of from 500 to 10,000 Pa·s at 25° C.

16. An oil-in-water emulsion formed in accordance with the method of claim 1.

17. A composition for personal care comprising the oil-in-water emulsion of claim 16 and at least one personal care ingredient.

18. A method of preparing a composition for personal care, said method comprising:
combining (A) an organopolysiloxane including at least two silicon-bonded hydrolysable or hydroxyl groups and (B) an organic oil to give a mixture;
combining the mixture, (C) an aqueous medium and (D) a surfactant to form an initial emulsion, wherein the mixture is a discontinuous phase in the aqueous medium (C);
contacting the organopolysiloxane (A) with an organic acid catalyst (D¹), which is the same as or different from the surfactant (D), to polymerize the organopolysiloxane (A) in the discontinuous phase of the initial emulsion to give a high molecular weight organopolysiloxane (A¹), thereby preparing an oil-in-water emulsion; and
combining the oil-in-water emulsion with at least one personal care ingredient.
